SEQUENCE LISTING (50 sequences)

SEQ ID NO: 1 - protein, 9 aa, Artificial Sequence
  Description of Artificial Sequence Synthetic peptide
CKGGRAKDC

SEQ ID NO: 2 - protein, 25 aa, Artificial Sequence
  Description of Artificial Sequence Synthetic peptide
CKGGRAKDCGGKLAKLAKKLAKLAK

SEQ ID NO: 3 - protein, 25 aa, Artificial Sequence
  Description of Artificial Sequence Synthetic peptide
CDKARGGKCGGKLAKLAKKLAKLAK

SEQ ID NO: 4 - protein, 25 aa, Artificial Sequence
  Description of Artificial Sequence Synthetic peptide
CKGGRAKDCGGKLAKLAKRLAKLAK

SEQ ID NO: 5 - protein, 29 aa, Artificial Sequence
  Description of Artificial Sequence Synthetic peptide
CKGGRAKDCGGKLAKLAKGRAKKLAKLAK

SEQ ID NO: 6 - protein, 25 aa, Artificial Sequence
  Description of Artificial Sequence Synthetic peptide
CKGGRAKDCGGKXAKXAKKXAKXAK

SEQ ID NO: 7 - protein, 25 aa, Artificial Sequence
  Description of Artificial Sequence Synthetic peptide
CKGGRAKDCGGKLAKLAKKLAKLAK

SEQ ID NO: 8 - protein, 7 aa, Artificial Sequence
  Description of Artificial Sequence Synthetic peptide
TRNTGNI

SEQ ID NO: 9 - protein, 7 aa, Artificial Sequence
  Description of Artificial Sequence Synthetic peptide
FDGQDRS

SEQ ID NO: 10 - protein, 6 aa, Artificial Sequence
  Description of Artificial Sequence Synthetic peptide
WGPKRL

SEQ ID NO: 11 - protein, 6 aa, Artificial Sequence
  Description of Artificial Sequence Synthetic peptide
WGESRL

SEQ ID NO: 12 - protein, 7 aa, Artificial Sequence
  Description of Artificial Sequence Synthetic peptide
VMGSVTG

SEQ ID NO: 13 - protein, 7 aa, Artificial Sequence
  Description of Artificial Sequence Synthetic peptide
KGGRAKD

SEQ ID NO: 14 - protein, 7 aa, Artificial Sequence
  Description of Artificial Sequence Synthetic peptide
RGEVLWS

SEQ ID NO: 15 - protein, 7 aa, Artificial Sequence
  Description of Artificial Sequence Synthetic peptide
TREVHRS

SEQ ID NO: 16 - protein, 7 aa, Artificial Sequence
  Description of Artificial Sequence Synthetic peptide
HGQGVRP

SEQ ID NO: 17 - protein, 9 aa, Artificial Sequence
  Description of Artificial Sequence Synthetic peptide
CKGGRAKDC

SEQ ID NO: 18 - protein, 9 aa, Artificial Sequence
  Description of Artificial Sequence Synthetic peptide
CKGGRAKDC

SEQ ID NO: 19 - protein, 9 aa, Artificial Sequence
  Description of Artificial Sequence Synthetic peptide
CDKARGGKC

SEQ ID NO: 20 - protein, 14 aa, Artificial Sequence
  Description of Artificial Sequence Synthetic peptide
KAAKKAAKAAKKAA

SEQ ID NO: 21 - protein, 21 aa, Artificial Sequence
  Description of Artificial Sequence Synthetic peptide
KLGKKLGKLGKKLGKLGKKLG

SEQ ID NO: 22 - protein, 14 aa, Artificial Sequence
  Description of Artificial Sequence Synthetic peptide
KLAKLAKKLAKLAK

SEQ ID NO: 23 - protein, 14 aa, Artificial Sequence
  Description of Artificial Sequence Synthetic peptide
KLAKLAKRLAKLAK

SEQ ID NO: 24 - protein, 18 aa, Artificial Sequence
  Description of Artificial Sequence Synthetic peptide
KLAKLAKGRAKKLAKLAK

SEQ ID NO: 25 - protein, 14 aa, Artificial Sequence
  Description of Artificial Sequence Synthetic peptide
KXAKXAKKXAKXAK

SEQ ID NO: 26 - protein, 14 aa, Artificial Sequence
  Description of Artificial Sequence Synthetic peptide
KLAKLAKKLAKLAK

SEQ ID NO: 27 - protein, 25 aa, Artificial Sequence
  Description of Artificial Sequence Synthetic peptide
CKGGRAKDCGGKLAKLAKKLAKLAK

SEQ ID NO: 28 - protein, 25 aa, Artificial Sequence
  Description of Artificial Sequence Synthetic peptide
CKGGRAKDCGGKLAKLAKKLAKLAK

SEQ ID NO: 29 - protein, 14 aa, Artificial Sequence
  Description of Artificial Sequence Synthetic peptide
KLAKLAKKLAKLAK

SEQ ID NO: 30 - protein, 25 aa, Artificial Sequence
  Description of Artificial Sequence Synthetic peptide
CKGGRAKDCGGKLAKLAKKLAKLAK

SEQ ID NO: 31 - protein, 14 aa, Artificial Sequence
  Description of Artificial Sequence Synthetic peptide
KLAKLAKKLAKLAK

SEQ ID NO: 32 - protein, 14 aa, Artificial Sequence
  Description of Artificial Sequence Synthetic peptide
KLAKLAKKLAKLAK

SEQ ID NO: 33 - protein, 14 aa, Artificial Sequence
  Description of Artificial Sequence Synthetic peptide
KLAKKLAKLAKKLA

SEQ ID NO: 34 - protein, 14 aa, Artificial Sequence
  Description of Artificial Sequence Synthetic peptide
KAAKKAAKAAKKAA

SEQ ID NO: 35 - protein, 21 aa, Artificial Sequence
  Description of Artificial Sequence Synthetic peptide
KLGKKLGKLGKKLGKLGKKLG

SEQ ID NO: 36 - protein, 7 aa, Artificial Sequence
  Description of Artificial Sequence Synthetic peptide
KLAKLAK

SEQ ID NO: 37 - protein, 25 aa, Artificial Sequence
  Description of Artificial Sequence Synthetic peptide
CKGGRAKDCGGKLAKLAKKLAKLAK

SEQ ID NO: 38 - protein, 7 aa, Artificial Sequence
  Description of Artificial Sequence Synthetic peptide
KLAKLAK

SEQ ID NO: 39 - protein, 25 aa, Artificial Sequence
  Description of Artificial Sequence Synthetic peptide
CDKARGGKCGGKLAKLAKKLAKLAK

SEQ ID NO: 40 - protein, 7 aa, Artificial Sequence
  Description of Artificial Sequence Synthetic peptide
KGGRAKD

SEQ ID NO: 41 - protein, 7 aa, Artificial Sequence
  Description of Artificial Sequence Synthetic peptide
DKARGGK

SEQ ID NO: 42 - protein, 25 aa, Artificial Sequence
  Description of Artificial Sequence Synthetic peptide
CKGGRAKDCGGKLAKLAKRLAKLAK

SEQ ID NO: 43 - protein, 21 aa, Artificial Sequence
  Description of Artificial Sequence Synthetic peptide
CNGRCGGKLAKLAKKLAKLAK

SEQ ID NO: 44 - protein, 29 aa, Artificial Sequence
  Description of Artificial Sequence Synthetic peptide
CKGGRAKDCGGKLAKLAKGRAKKLAKLAK

SEQ ID NO: 45 - protein, 4 aa, Artificial Sequence
  Description of Artificial Sequence Synthetic peptide
GRAK

SEQ ID NO: 46 - protein, 25 aa, Artificial Sequence
  Description of Artificial Sequence Synthetic peptide
CKGGRAKDCGGKXAKXAKKXAKXAK

SEQ ID NO: 47 - protein, 14 aa, Artificial Sequence
  Description of Artificial Sequence Synthetic peptide
KXAKXAKKXAKXAK

SEQ ID NO: 48 - protein, 25 aa, Artificial Sequence
  Description of Artificial Sequence Synthetic peptide
CKGGRAKDCGGKLAKLAKKLAKLAK

SEQ ID NO: 49 - protein, 25 aa, Artificial Sequence
  Description of Artificial Sequence Synthetic peptide
CKGGRAKDCGGKLAKLAKKLAKLAK

SEQ ID NO: 50 - protein, 15 aa, Artificial Sequence
  Description of Artificial Sequence Synthetic peptide
GKLAKLAKKLAKLAK

(19) United States
(12) Patent Application Publication
(10) Pub. No.: US 2013/0237476 A1
(43) Pub. Date: Sep. 12, 2013

(54) ADIPOSE TISSUE TARGETED PEPTIDES

(71) Applicant: ABLARIS THERAPEUTICS INC., Pasadena, CA (US)

(72) Inventor: James F. HULVAT, Kalispell, MT (US)

(73) Assignee: ABLARIS THERAPEUTICS INC., Pasadena, CA (US)

(21) Appl. No.: 13/785,566

(22) Filed: Mar. 5, 2013

Related U.S. Application Data
(60) Provisional application No. 61/608,389, filed on Mar. 8, 2012.

(51) Int. Cl. C07K 14/00; C07K 7/02

(52) U.S. Cl. CPC C07K 14/00; C07K 7/02
USPC 514/4.8; 530/324; 530/323; 514/21.3; 514/21.5

(57) ABSTRACT

The application provides synthetic peptide conjugates capable of targeting and causing ablation of adipose tissue in mammal comprising at least one targeting peptide and at least one therapeutic peptide. The synthetic peptide conjugates are envisaged to have decreased physiological toxicity and/or enhanced in situ cytotoxicity compared to the peptide CKGGRAKDC-GG-_{D}(KLAKLAKKLAKLAK) (SEQ ID NO: 2).

### CROSS-REFERENCE TO RELATED APPLICATIONS

The present application claims the benefit of U.S. Provisional Patent Application No. 61/608,389, filed Mar. 8, 2012, the disclosure of which is incorporated by reference herein in its entirety.

The present application contains a Sequence Listing which has been submitted in ASCII format via EFS-Web and is hereby incorporated by reference in its entirety. Said ASCII copy, created on Feb. 28, 2013, is named 0052-AB02-US1_SL.txt and is 20,539 bytes in size.

### FIELD OF THE INVENTION

The present application relates to the fields of molecular medicine and targeted delivery of therapeutic agents. More specifically, the present application also relates to compositions that selectively target adipose tissue.

### BACKGROUND OF THE INVENTION

Obesity is an increasingly prevalent human condition in developed societies. Despite major progress in the understanding of the molecular mechanisms leading to obesity, no safe and effective pharmacological treatment has yet been found.

Targeting peptides that exhibit selective and/or specific binding for adipose tissues have been previously reported (see e.g., U.S. Pat. No. 7,452,964). Targeting peptides against adipose tissues would have a variety of potential uses, e.g., to control obesity and related conditions. Adipose-targeting peptides would also be of potential use to treat HIV related adipose malformations such as lipodystrophia and/or hyperlipidemia (see, e.g., Zhang et al. J. Clin. Endocrin. Metab, 84:4274-77, 1999; Jain et al., Antiviral Res. 51:151-1.77, 2001; Raolin et al., Prog. Lipid Res. 41:27-65, 2002).

Presently available methods for control of weight include lifestyle modification and various forms of bariatric surgery. Diet and exercise programs often fail to produce significant long-term weight loss, and surgical intervention is both costly and can result in serious complications. Dieting includes both popular (fad) diets and the use of dietary supplements and appetite suppressants. These approaches rarely achieve long-term weight control, and are often unhealthy, since important nutrients may be missing from the diet. After losing weight, the dieters typically return to their original eating habits. This often results in weight gain that can exceed the subject's weight before dieting the “yo yo effect”).

Appetite suppressant drugs such as Phentermen HCl, Meridia, Xernical, Adipex-P, Bontril and Ionomin may have adverse effects, such as addiction, dry mouth, nausea, irritability, and constipation. Placebo-controlled clinical trials of the available, FDA approved drugs for obesity demonstrate only limited weight loss is achieved (GA, Kennett and P. G. Clifton Pharmacol. Biochem. & Behavior 97 (2010) 6343). Effective drugs for controlling weight, such as fenfluramine, were withdrawn from the market due to cardiotoxicity, and others anti-obesity drugs recently submitted for FDA approval have met with rejection due to safety concerns. [A, Pollack, New York Times, Feb. 2, 2011, p. B1]

Surgical methods for weight reduction, such as liposuction and gastric bypass surgery, have many risks. Liposuction removes subcutaneous fat through a suction tube inserted into a small incision in the skin. Risks and complications may include scarring, bleeding, infection, change in skin sensation, pulmonary complications, skin loss, chronic pain, etc. In gastric bypass surgery, the patient has to go through the rest of his or her life with a drastically altered diet due to the reduction in stomach capacity. Side effects may include nausea, diarrhea, bleeding, infection, bowel blockage caused by scar tissue, hernia and adverse reactions to general anesthesia. The most serious potential risk is leakage of fluid from the stomach or intestines, which may result in abdominal infection and the need for a second surgery. None of the presently available methods for weight control is satisfactory.

Another adipose related disease state is lipodystrophy syndrome(s) related to HIV infection (e.g., Jain et al., Antiviral Res. 51:151-177, 2001). Mortality rates from HIV infection have decreased substantially following use of highly active antiretroviral therapy (HAART) (Id.) However, treatment with protease inhibitors as part of the HAART protocol appears to result in a number of lipid-related symptoms, such as hyperlipidemia, fat redistribution with accumulation of abdominal and cervical fat, diabetes mellitus and insulin resistance (Jain et al., 2001; Yanovski et al., J. Clin. Endocrin. Metab, 84:19254931; Raulin et al., Prog, Lipid Res. 41:27-65, 2002). Although of minor significance compared to the underlying HIV infection and possible development of AIDS related complex (ARC) and/or AIDS, lipodystrophy syndrome adversely affects quality of life and may be associated with increased risk of coronary artery disease, heart attack, stroke and other adverse side-effects of increased blood lipids. While treatment with metformin, an insulin-sensitizing agent, has been reported to provide some alleviation of symptoms (Hadi an et al., J. Amer. Med. Assn. 284:472477, 2000), more effective methods of treating HIV related lypodystrophy are desired.

Antiobesity therapy based on targeted induction of apoptosis in the vasculature of adipose tissue has also been described. Kolonin et al., Nat Med. 2004 June; 10(6):625-32. Epub 2004 May 9, showed that the CKGGRAKDC (SEQ ID NO: 1) targeting peptide associates with prohibitin, a multifunctional membrane-associated protein, mitochondrial membrane chaperone and transmembrane signaling receptor expressed in adipose tissue.

The synthetic peptide conjugate (CKGGRAKDC-GG-_{D}(KLAKLAKKLAKLAK) (SEQ ID NO: 2; also referred to herein as “ABL-1”) contains the targeting peptide operably linked to an apoptotic therapeutic peptide and is able to target prohibitin expressed in the adipose vascular endothelial cells and cause ablation of visceral adipose tissue (termed “white fat”). Resorption of established white fat resulted in normalization of metabolism and rapid obesity reversal in animal models. The apoptotic therapeutic peptide sequence was originally described in M. M. Javadpour et al., J. Med. Chem. (1996), 39(16), 3107-3113. It is known to disrupt mitochondrial membranes upon receptor-mediated cell internalization and the D-enantiomer is resistant to proteolysis by peptidases in blood plasma.

Compounds containing the apoptotic _{D}(KLAKTAKIKLAKLAK) (SEQ ID NO:26) peptide may be associated with physiological toxicity. (K. Karialainen et al. Blood (2001)117:3, 920-927). Published reports indicate limited in vitro cytotoxicity of this sequence due to the inability of the highly positively charged amino acids to penetrate eukaryotic plasma membranes. (H. M. Ellerby, Nat. Med. (1999) 5:9, 1032) However, in vivo metabolites of D-amino acid peptides are cleared by the kidneys, where they may be concentrated and could exhibit renal toxicity. Moreover, the L-amino acid structure of the SEQ ID NO: 1 targeting peptide makes it susceptible to rapid proteolysis relative to the apoptotic _{D}(KLAKLAKKLAKLAK) (SEQ ID NO:26) peptide, reducing the overall targeting efficiency and potency of the ABL-1 peptide conjugate. As such, there remains a need to identify peptides capable of targeting and ablating adipose tissue which are not associated any with physiological toxicity but have enhanced potency relative to ABL-1.

### SUMMARY OF THE INVNETION

One aspect of the invention relates to synthetic peptide conjugates capable of targeting and causing reduction of adipose tissue in a mammal comprising at least one targeting peptide and at least one therapeutic peptide. The synthetic peptide conjugates of the invention can have 1, 2, 3, 4, 5, 6, 7, 8, 9, 10 or more of either or both of the targeting or therapeutic peptides.

In one embodiment the synthetic peptide conjugates are selected from the group consisting of _{D}(CKGGRAKDC)-GG-_{D}(KLAKLAKKLAKLAK) (SEQ ID NO: 28); _{D}(C)KARGGKC)-GG-_{D}(KLAKLAKKLAKLAK) (SEQ ID NO: 3); CKGGRAKDC-GG-_{D}(KLAKLAK)-RL-_{D}(AKLAK) (SEQ ID NO: 4); CKOGRAKDC-GG-_{D}(KLAKLAK)-GRAK-_{D}(KLAKLAK) (SEQ ID NO: 5); CKGGRAKDC-GG-_{D,L}-(KFxAKFxAKKFxAKFxAK) (wherein Fx can be a cyclohexylalanine) (SEQ ID NO: 6); CKGGRAKDC-G-(PEG-G-_{D}(KLAKLAKKLAKLAK) (SEQ ID NO: 7); and (PEG)ₙ-CKGGRAKDC-GG-_{D}(KIAKIAKKLAKLAK) (SEQ ID NO: 27). (wherein (PEG), is an oligomer of ethylene glycol of the form —O(CH₂CH₂O)ₙ— and n is an integer ranging from 4 to 30, or (PEG)ₙ is a polymer of polyethylene glycol) with an average molecular weight of up to about 40,000 Da.

In another embodiment, the targeting peptide is TRNTGNI (SEQ ID NO:8), FDGQDRS (SEQ ID NO:9); WGPKRI, (SEQ ID NO:10); WGESRL (SEQ ID NO:11); VMGSVTG (SEQ ID NO: 12); KGGRAKD (SEQ ID NO:13); RGEVLWS (SEQ ID NO:14); TREWIRS (SEQ ID NO: 15); 1-10QGVRP (SEQ ID NO:16); CKGGIRAKDC (SEQ ID NO: 17); or substantially similar variants thereof. In one embodiment, the substantially similar variants have an endopeptidase cleavage site. In another embodiment, alkylation of amines, such as N-methyl glycine (sarcosine) are used in place of one or more L-amino acids to limit endopeptidase cleavage. In another embodiment, the substantially similar variants have a reduction in the number of overall positively charged amino acids relative to their reference sequence. In one embodiment, the targeting peptide contains all or only 1, 2, 3, 4, 5, 6, 7 or more D-amino acids. In another embodiment, the targeting peptide contains all or only 1, 2, 3, 4, 5, 6, or 7 L-amino acids.

In a further embodiment the targeting peptide is _{D}C-_{D}K-G-G-_{D}R-_{D}A-_{D}K-_{D}D-_{D}C (SEQ ID NO 18).

In another embodiment, the targeting in peptide is _{D}C-_{D}D-_{D}K-_{D}A-_{D}R-G-G-_{D}K-_{D}C (SEQ ID NO: 19).

In another embodiment, the therapeutic peptide has a site susceptible to hydrolytic cleavage e.g., an endopeptidase cleavage site. In another embodiment the therapeutic peptide has a helical structure and comprises an additional three to four amino acids to provide for an additional helical torn.

In another embodiment, the targeting peptide is cyclical and the amino acid sequence is modified so as to prevent its proteolytic degradation.

In a further embodiment of this aspect of the invention, the synthetic peptide conjugates comprise one or more polymer molecules. The polymer may for example be polyethylene polymer, e.g., polyethylene glycol (“PEG”). The PEG could be PEG 100 (100 molecular weight (MW)), PEG 200, PEG 300, PEG 400, PEG 500, PEG 600, PEG 700, PEG 800, PEG 900, PEG 1000, PEG 1100, PEG 1200, PEG 1300, PEG 1400, PEG 1500 PEG 1600, PEG 1700, PEG 1800, PEG2000, PEG 3000, PEG 4000, PEG 5000, PEG 6000, PEG 7000, PEG 5000, PEG 5000, PEG 10,000, PEG 15,000, PEG 20,000, PEG 25,000, PEG 30,000, PEG 35,000, PEG 40,000, or mixtures thereof, and molecular weights between these values. The polymer may be attached to the N- and/or C-terminus of the peptide and/or intermediate to the targeting and therapeutic peptides and/or on one or more internal amino acid residues of either peptide. Additionally, the polymer may be used as a spacer to link the targeting and therapeutic peptides.

In another embodiment, the therapeutic peptide is capable of inducing apoptosis and removal of adipose tissue (i.e., ablation). In one embodiment the therapeutic peptide is KLAKLAKKLAKLAK (SEQ ID NO:29), (KLAKKLA)₂ (SEQ ID NO:33), (KAAKKAA)₂ (SEQ ID NO:20) or (KLGKKLG)₃ (SEQ ID NO:21) or a peptide substantially similar thereto. In one embodiment, the therapeutic peptide contains all or only 1, 2, 3, 4, 5, 6, 7 or more D-amino acids. In another embodiment, the therapeutic peptide contains all or only 1, 2, 3, 4, 5, 6, 7 or more D-amino acids. In another embodiment, the therapeutic peptide is _{D}K-_{D}L-_{D}A-_{D}K-_{D}L-_{D}A-_{D}K-_{D}K-_{D}L-_{D}A-_{D}K-_{D}L-_{D}-_{D}K (SEQ ID NO: 22). In another embodiment, the therapeutic peptide is _{D}K-_{D}L-_{D}A-_{D}K-_{D}L-_{D}A-_{D}K-_{D}R-_{D}L-_{D}A-_{D}K-_{D}L-_{D}A-_{D}K ((SEQ ID NO: 23). In a further embodiment, the therapeutic peptide is _{D}K-_{D}L-_{D}A-_{D}K-_{D}L-_{D}A-_{D}K-G-_{L}R-_{L}A-_{L}K-_{D}K-_{D}L-_{D}A-_{D}K-_{D}L-_{D}A-_{D}K (SEQ ID NO: 24). In still another embodiment, the therapeutic peptide is _{D}K-_{D}Fx-_{D}A-_{D}K-_{D}Fx-_{D}A-_{D}K-_{D}K-_{D}Fx-_{D}K-_{D}K-_{D}Fx-_{D}A-_{D}K (SEQ ID NO: 25) wherein the Fx is a modified or non-natural amino acid, e.g., cyclohexylalanine.

In a further embodiment, the targeting peptide and the therapeutic peptide are joined through a linker. The linker may act through covalent or non-covalent interactions, e.g., hydrophobic, ionic or hydrogen bonds. The linker may be 1, 2, 3, 4, 5, 6, 7, 8, 9 10 or more amino acids in length. Alternatively, the linker may be a polymer such as a PEG.

Another aspect of the invention relates to methods of treating obesity and/or a metabolic disorder in a patient comprising providing a patient in need thereof with a therapeutically effective amount of the synthetic peptide conjugates described herein. In one embodiment, the synthetic peptide conjugate is selected from the group consisting of

(SEQ ID NO: 28)
_{D}(CKGGRAKDC)-GG-_{D}(KLAKLAKKLAKLAK);
(SEQ ID NO: 3)
_{D}(CDKARGGKC)-GG-_{D}(KLAKLAKKLAKLAK);
(SEQ ID NO: 4)
CKGGRAKDC-GG-_{D}(KLAKLAK)-RL-_{D}(AKLAK);
(SEQ ID NO: 5)
CKGGRAKDC-GG-_{D}(KLAKLAK)-GRAK-_{D}(KLAKLAK);
(SEQ ID NO: 6)
CKGGRAKDC-GG-_{D,L}-(KFxAKFxAKKFxAKFxAK)
(wherein Fx can be a cyclohexylalanine);
and
(SEQ ID NO: 30)
CKGGRAKDC-G-(PEG)₂₇-G-_{D}(KLAKLAKKLAKLAK).

Another aspect of this application relates to methods of determining whether the synthetic peptide conjugates or substantially similar variants thereof are suitable for treating obesity and/or a metabolic disease comprising contacting the proteins with adipose vascular endothelial cells and determining whether the protein selectively binds the cells. In a further embodiment, the methods involve determining whether the vascular endothelial cells become apoptotic following contact with the synthetic peptide conjugates. Suitable assays for carrying out the methods set forth in this aspect of the application may be found in Kolonin et al., Nature Medicine, 2004 which is expressly incorporated by reference in its entirety.

### BRIEF DESCRIPTION OF THE FIGURES

A more complete understanding of the present invention may be obtained by reference to the accompanying drawings, when considered in conjunction with the subsequent detailed description. The embodiments illustrated in the drawings are intended only to exemplify the invention and should not be construed as limiting the invention to the illustrated embodiments, in which:

FIG. 1 shows the structure of the ABL-1 (SEQ ID NO:2). (Kolonin et al., Nature Medicine, 2004).

FIG. 2 shows the amphipathic helical structure of the KLAKLAKIKLAKLAK (SEQ ID NO:29) targeting peptide. (L. A. Plesniak et al. Protein Sci. (2004), 13, 19813-1996)

### DETAILED DESCRIPTION OF THE INVENTION

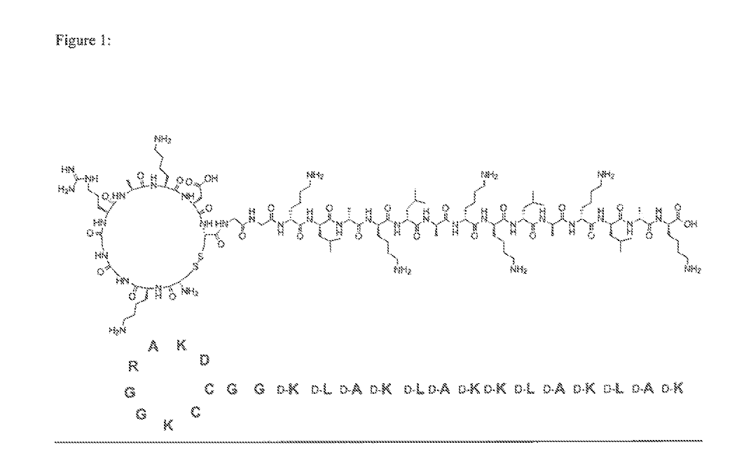

This application relates to synthetic peptide conjugates which are envisaged to be associated with increased therapeutic activity relative to ABL-1 (see FIG. 1) and are associated with lower physiological toxicity relative to ABL-1. These improvements provide to a greater therapeutic window of the inventive therapeutic proteins relative to ABL-1.

The synthetic peptide conjugates disclosed herein are envisaged to have increased stability of targeting peptides relative to ABL-1. For example, the ABL-1 peptide's targeting peptide is cyclic. The synthetic peptide conjugates disclosed herein have modified amino acid sequences of their targeting peptides to enhance their resistance to proteolytic degradation.

Alternatively, the synthetic peptide conjugates are envisaged to have increased therapeutic efficacy relative to ABL-1 sequence. For example, the application envisages enhancing the apoptotic potency of the _{D}(KLAKLAK)₂ (SEQ ID NO:31), apoptotic therapeutic peptide.

Alternatively the synthetic peptide conjugates are envisaged to have improved renal clearance due to the incorporation of endopeptidase cleavage sites within the therapeutic peptide.

As used herein in the specification, “a” or “an” may mean one or more. As used herein in the claim(s), in conjunction with the word “comprising,” the words “a” or “an” may mean one or more than one. As used herein “another” may mean at least a second or more of an item.

A “targeting peptide” is a peptide comprising a contiguous sequence of amino acids, which is characterized by selective localization to an organ, tissue or cell type in general and adipose tissue or cells in particular. A targeting peptide is considered to be selectively localized to a tissue or organ if it exhibits greater binding in that tissue or organ compared to a control tissue or organ. Preferably, selective localization of a targeting peptide should result in a two-fold or higher enrichment of the peptide in the target organ, tissue or cell type, compared to a control organ, tissue or cell type. Selective localization resulting in at least a three-fold, four-fold, five-fold, six-fold, seven-fold, eight-fold, nine-fold, ten-fold or higher enrichment in the target organ compared to a control organ, tissue or cell type is more preferred. Alternatively, a targeting peptide that exhibits selective localization preferably shows an increased enrichment in the target organ compared to a control, “Targeting peptide” and “horning peptide” are used synonymously herein.

The synthetic peptide conjugates have a “decreased physiological toxicity” relative to ABL-1. Physiological toxicity includes renal toxicity. Renal toxicity may be a general characteristic of compounds containing the _{D}[KLAKLAK], (SEQ ID NO:31) apototic peptide sequence. While not wishing to be bound by any particular theory, renal toxicity could result from uptake and reabsorption of apoptosis inducing peptides by renal proximal tubule cells. The low molecular weight (2555 g/mol) of the ABL-1 peptide indicates the peptide is likely taken up by endosomes in the brush boarder of the kidneys and broken down via renal clearance mechanisms. Metabolites of ABL-1 are likely to retain the _{D}(KLAKLAK)₂ (SEQ ID NO:31) apototic peptide, as D-amino acids are known to resist proteolytic degradation. As such, renal toxicity may for example be measured by the amount of time the synthetic peptide conjugates or their metabolites remain in the serum following administration (e.g., half-life) or the rate at which any of the synthetic peptide conjugates or their metabolites accumulate in the urine of patients over time.

Decreased physiological and/or renal toxicity of the synthetic peptide conjugates is envisaged to be at least 10%, 15%, 20%, 25%, 30%, 35%, 40%, 45%, 50%, 55%, 60%, 65%, 70%, 75%, 80%, 90%, 95% or 100% relative to ABL-1. The synthetic peptide conjugates may have at least a three-fold, four-fold, five-fold, six-fold, seven-fold, eight-fold, nine-fold, ten-fold or higher reduction in physiological toxicity relative to ABL-1.

The synthetic peptide conjugates are envisaged to have increased therapeutic activity relative to ABL-1. Therapeutic activity can include apoptotic activity in adipose vascular endothelial cells in culture and/or at the site of action (i.e., in situ) in adipose tissues. The apoptotic process of programmed cell death leads to characteristic cell changes (morphology) and death. These changes include membrane blebbing, loss of cell membrane asymmetry and attachment, cell shrinkage, nuclear fragmentation, chromatin condensation, and chromosomal DNA fragmentation. Unlike necrosis, apoptosis produces cell fragments called apoptotic bodies that surrounding cells are able to engulf and quickly remove before the contents of the cell can spilt out onto surrounding cells and cause damage. Such apoptotic activity can be determined through standard apoptotic assays well known in the art, such as for example caspase assays, TUNEL and DNA fragmentation assays, cell permeability assays, annexin V assays, protein cleavage assays, mitochondrial ATP/ADP assays, and acridine orange staining.

Therapeutic activity can also be determined by a decrease in adipose tissue in a mammal through, e.g., fat resorption. As such, therapeutic activity measurements involve measures of body fat. An individual's body fat percentage is the total weight of an individual's fat divided by their weight and consists of essential body fat and storage body fat. This may be determined by well-known assays including weight, body-mass index, skin fold measurements or body fat percentage measurements through, e.g., volume displacement, bioelectrical impedance analysis, near-infrared interactance, dual energy X-ray absorptiometry and body average density measurement.

To test the therapeutic activity of the synthetic peptide conjugates, well-known in vivo models of obesity may be used. For example, assays for determining liver fat content, serum leptin levels, adipocyte counts, serum ketone body (e.g., acetoacetate and 3-β-hydroxybutyrate) levels may be used. Additionally, metabolic assays may also be used to determine therapeutic activity relating to adipose tissue ablation by e.g., measuring oxygen consumption, carbon dioxide production, heat generation, and spontaneous locomotor activity, blood glucose levels and/or insulin levels/tolerance. Additionally, Lep^{ob/ob} mice may be utilized.

Therapeutic activity of the synthetic peptide conjugates may also be measured by a reduction in serum cholesterol or triglyceride levels, a reduction in appetite or a reduction in symptoms associated with diabetes or other metabolic disorders (e.g., blood glucose levels, insulin resistance).

Increased therapeutic activity of the synthetic peptide conjugates is envisaged to be at least 10%, 15%, 20%, 25%, 30%, 35%, 40%, 45%, 50%, 55%, 60%, 65%, 70%, 75%, 50%, 90%, 95% or 100% relative to ABL-1. The synthetic peptide conjugates may have at least a three-fold, tour-fold, five-fold, six-fold, seven-fold, eight-fold, nine-fold, ten-fold or higher increase in therapeutic activity to ABL-1.

Increased stability of the targeting peptides of the synthetic peptide conjugates relative to ABL-4 means that the targeting peptides are not as readily metabolized as the targeting peptide of ABL-1. The stability might for example, result from the use of modified amino acids and/or the removal of certain known enzymatic cleavage sites, e.g. endopeptidase cleave sites.

Increased stability of the targeting peptides of the synthetic peptide conjugates is envisaged to be at least 10%, 15%, 20%, 25%, 30%, 35%, 40%, 45%, 50%, 55%, 60%, 65%, 70%, 75%, 80%, 90%, 95% or 100% relative to ABL-1. The synthetic peptide conjugates may have at least a three-fold, four-fold, five-fold, six-fold, seven-fold, eight-fold, nine-fold, ten-fold or higher increase in targeting peptide stability relative to ABL-1.

Exemplary targeting peptides that selectively localize to adipose tissue include: TRNTGNI (SEQ ID NO:8); FDGQDRS (SEQ ID NO:9); WGPKRL (SEQ ID NO:10); WGESRL (SEQ ID NO:11); VMGSVTG (SEQ ID NO:12); KGGRAKD (SEQ ID NO:13); RGEVLWS (EQ ID NO:14); TREVHRS (SEQ ID NO:15); HGQGVRP (SEQ ID NO:16); CIKOGRAKDC (SEQ ID NO:17); and substantially similar variants thereof.

A “receptor” for a targeting peptide includes but is not limited to any molecule or macromolecular complex that binds to a targeting peptide. Non-limiting examples of receptors include peptides, proteins, glycoproteins, lipoproteins, epitopes, lipids, carbohydrates, multi-molecular structures, a specific conformation of one or more molecules and a morphoanatomic entity. In some embodiments, a “receptor” is a naturally occurring molecule or complex of molecules that is present on the lumenal surface of cells forming blood vessels within a target organ, tissue or cell type. In the preferred embodiment, the receptor is the prohibitin.

In embodiments, compositions are provided comprising at least one peptide. As used herein, peptide generally refers, but is not limited to, a sequence of greater than about 200 amino acids, up to a full length sequence translated from a gene; a sequence of greater than about 100 amino acids; and/or a sequence of from about 3 to about 100 amino acids. For convenience, the terms “protein,” “polypeptide” and “peptide” are used interchangeably herein.

In certain embodiments the size of at least one peptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 31, 82, 83, 84, 85, 36, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 300, about 825, about 850, about 875, about 900, about 925, about 950, about 975, about 1000, about 1100, about 1200, about 1300, about 1400, about 1500, about 1750, about 2000, about 2250, about 2500 or greater amino acid residues.

As used herein, an “amino acid residue” refers to any naturally occurring amino acid, any amino acid derivative or any amino acid mimic known in the art. In certain embodiments, the residues of the peptide are sequential, without any non-amino acid interrupting the sequence of amino acid residues. In other embodiments, the sequence may comprise one or more non-amino acid moieties, particular embodiments, the sequence of residues of the peptide may be interrupted by one or more non-amino acid moieties. Accordingly, the term “peptide” encompasses amino acid sequences comprising at least one of the 20 common amino acids found in naturally occurring proteins, or at least one modified or unusual amino acid, including but not limited to those shown on Table 1 below.

TABLE I
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | Ide | Isodesmosine |
| Acp | 6-Aminocaproic acid | AIle | allo-Isoleucine |
| Ahe | 2-Aminoheptanoic acid | MeGly | N-Methylglycine, |
| Aib | 2-Aminoisobutyric acid | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

Peptides described herein may be made by any technique known to those of skill in the art, including the expression through standard molecular biological techniques, isolation from natural sources, or chemical synthesis. Suitably, the synthetic peptide conjugates are produced via chemical synthesis as described herein and otherwise known in the art.

The nucleotide and protein, polypeptide and peptide sequences corresponding to various genes have been previously disclosed, and may be found at computerized databases known to those of ordinary skill in the ant. One such database is the National Center for Biotechnology Information's Genbank and GenPept databases (world wide web at nbci.nlm.nih.gov/). The coding regions for known genes may be amplified and/or expressed using the techniques disclosed herein or as would be known to those of ordinary skill in the art. Alternatively, various commercial preparations of peptides are known to those of skill in the art.

Peptides “substantially similar” to a given reference amino acid sequence described herein refers to a peptides which have substantially similar or the same functional, e.g., targeting, attributes as the referenced amino acid sequence but vary with respect to amino acid sequence. Such variation could be the result of the addition, substitution and/or deletion of 1, 2, 3, 4, 5, 6, 7, 8, 9, 10, 11-15, 16-20 or more amino acid residues relative to the reference sequence. Such peptides will, therefore, be 99%, 98%, 97%, 96%, 95%, 94%, 93% 92%, 91%, 90%, 89%, 88%, 87%, 86%, 85%, 84%, 83%, 82%, 81%, 30%, 79%, 78%, 77%, 76% or 75% identical to the reference sequence. Reviewing the disclosure herein would provide the skilled artisan with sufficient information as to which additions, substitutions, deletions and/or modifications would be appropriate to obtain a substantially similar peptide variant that retains the same or substantially similar functional, e.g., targeting and/or therapeutic attributes as the referenced amino acid sequence. Appropriate substitutions are for example, making conservative substitutions of similarly hydrophilic, hydrophobic, or charged amino acids; and/or addition or removal of leader sequences. The nucleic acids encoding a reference peptide will preferably hybridize under high stringency conditions to the complement of a nucleic acid encoding a peptide substantially similar to the reference peptide.

Another embodiment for the preparation of peptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

Other embodiments of the present invention concern synthetic peptide conjugates. These molecules generally have all or a substantial portion of a targeting peptide, linked at the N- or C-terminus, to all or a portion of a second peptide. The second peptide will preferably have a therapeutic function and work through a mechanism of action such as e.g., inducing apoptosis. For example, synthetic peptide conjugates may employ leader sequences from other species to permit the recombinant expression of a protein in a heterologous host. Another useful conjugate includes the addition of an immunologically active domain, such as an antibody epitope, to facilitate purification of the synthetic peptide conjugates. Inclusion of a cleavage site at or near the conjugation will facilitate removal of the extraneous peptide after purification. Other useful congutaes include linking of functional domains, such as active sites from enzymes, glycosylation domains, cellular targeting signals or transmembrane regions.

In preferred embodiments, the synthetic peptide conjugates comprise a targeting peptide linked to a therapeutic protein or peptide. Examples of proteins or peptides that may be incorporated into a synthetic peptide conjugate include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually any protein or peptide could be incorporated into a synthetic peptide conjugate comprising a targeting peptide. Methods of generating synthetic peptide conjugates are well known to those of skill in the art. Such proteins can be produced, for example, by chemical attachment using bifunctional cross-linking reagents, by de novo synthesis of the complete fusion peptide, or by attachment of a DNA sequence encoding the targeting peptide to a DNA sequence encoding the second peptide or protein, followed by expression of the intact synthetic peptide conjugate.

In certain embodiments a protein or peptide may be isolated or purified. Protein purification techniques are well known to those of skill in the art. These techniques involve, at one level, the homogenization and crude fractionation of the cells, tissue or organ to peptide and non-peptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. A particularly efficient method of purifying peptides is fast performance liquid chromatography (FPLC) or even high performance liquid chromatography (HPLC).

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind. This is a receptor ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

Because of their relatively small size, the targeting peptides described herein can be synthesized in solution or on a solid support in accordance with conventional techniques. Various automatic synthesizers are commercially available and can be used in accordance with known protocols. See, for example, Stewart and Young, Solid Phase Peptide Synthesis, 2d ed. Pierce Chemical Co, 1984; Tam et al., J. Am. Chem. Soc., 105:6442, 1983; Merrifield, Science, 232: 341-347, 1986; and Barmy and Merrifield. The Peptides, Gross and Meienhofer, eds., Academic Press, New York, pp. 1-284, 1979, each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

In certain embodiments, it may be desirable to couple specific bioactive agents and/or therapeutic peptides to one or more targeting peptides for targeted delivery of the synthetic peptide conjugates to an organ, tissue or cell type. Such agents include, but are not limited to, cytokines, chemokines, pro apoptosis factors and anti-angiogenic factors. The term “cytokine” is a generic term for proteins released by one cell population that act on another cell as intercellular mediators.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian inhibiting substance; mouse gonadotropin-associated peptide; inhibin; activin; vascular endothelial growth factor; integrin; thrombopoietin (TPO); nerve growth factors such as NGF platelet-growth factor; transforming growth factors (TGFs) such as TGF-.alpha, and TGF-.beta; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3_{, IL-}4, IL-5, IL-6, IL-7, IL-8, IL-9, IL-11, I-12; 13, IL-14, IL-15, IL-16, IL-17, IL-18, LIF, GCSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

Chemokines generally act as to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta and IP-10. The skilled artisan will recognize that certain cytokines are also known to have chemoattractant effects and could also be classified under the term chemokines.

In certain embodiments, the synthetic peptide conjugates may be attached to imaging agents of use for imaging and diagnosis of various diseased organs, tissues or cell types. Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (ill), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III),

Radioisotopes of potential use as imaging or therapeutic agents include astatine²¹¹, ¹⁴-carbon, ⁵¹chromium, ³⁶chlorine, ⁵⁷cobalt, ⁵⁸cobalt, ° copper, ¹⁵²Eu, ⁶⁷gallium, ³hydrogen, ¹²³iodine, ¹³⁵ iodine, ¹³¹iodine, ¹¹¹indium, ⁵⁹iron, ³²phosphorus, ¹⁸⁶rhenium, ¹⁸⁸rhenium, ⁷⁵selenium, ³⁵sulphur, ⁹⁹technicium and ⁹⁰yttrium.

Radioactively labeled proteins or peptides may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides may be labeled with ^{99M}technetium by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups that are often used to bind radioisotopes that exist as metallic ions to peptides are diethylenetriminepenta-acetic acid (DTPA) and ethylene diaminetetra-acetic acid (EDTA). Also contemplated for use are fluorescent labels, including rhodainine, fluorescein isothiocyanate and renographin.

In certain embodiments, the claimed proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

Bifunctional crosslinking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites, Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol reactive group.

Exemplary methods for cross-linking ligands to delivery vehicles are described in U.S. Pat. No. 5,603,872, U.S. Pat. No. 5,401,511, and 7,270,808 (each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites are dictated by the liposome formulation and the liposome type. The liposomal surfaces may also have sites for non-covalent association. To form covalent conjugates of ligands and liposomes, cross-linking reagents have been studied for effectiveness and blocotupatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl)carbodiimicle (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups.

Nucleic acids as described herein may encode a targeting peptide, a receptor protein, a fusion protein or other protein or peptide. The nucleic acid may be derived from genomic DNA, complementary DNA (cDNA) or synthetic DNA. Where incorporation into an expression vector is desired, the nucleic acid may also comprise a natural intron or an intron derived from another gene. Such engineered molecules are sometime referred to as “mini-genes,”

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of almost any size, determined in part by the length of the encoded protein or peptide.

It is contemplated that targeting peptides, fusion proteins and receptors may be encoded by any nucleic acid sequence that encodes the appropriate amino acid sequence. The design and production of nucleic acids encoding a desired amino acid sequence is well known to those of skill in the art, using standardized codon tables (see Table 2 below). In preferred embodiments, the codons selected for encoding each amino acid may be modified to optimize expression of the nucleic acid in the host cell of interest. Codon preferences for various species of host cell are well known in the art.

TABLE 2

| Amino Acid | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | P | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |

In addition to nucleic, acids encoding the desired peptide or protein, also included are complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

In certain embodiments expression vectors are employed to express the targeting peptide or fusion protein, which can then be purified and used. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are known.

The terms “expression construct” or “expression vector” are meant to include any type of genetic construct containing a nucleic acid coding for a gene product in which part or all of the nucleic acid coding sequence is capable of being transcribed. In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent and under the control of a promoter that transcriptionally active in human cells. Generally speaking, such a promoter might include either a human or viral promoter.

In various embodiments, the human cytomegalovirus (CMV) immediate early gene promoter, the SV40 early promoter, the Rouse sarcoma virus long terminal repeat, rat insulin promoter, and glyceraldehyde-3-phosphate dehydrogenase promoter can be used to obtain high-level expression of the coding sequence of interest. The use of other viral or mammalian cellular or bacterial phage promoters that are well-known in the art to achieve expression of a coding sequence of interest is contemplated as well, provided that the levels of expression are sufficient for a given purpose.

Where a cDNA insert is employed, one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome, and express viral genes stably and efficiently have made them attractive candidates for the transfer of foreign genes into mammalian cells (Ridgeway, 1988; Nicolas and Rubinstein, In: Vectors: A survey of molecular cloning vectors and their uses, Rodriguez and Denhardt, eds., Stoneham: Butterworth, pp. 494-513, 198&; Baichwal and Sugden, Baichwal, In: Gene Transfer, Kucherlapati R, ed., New York, Plenum Press, pp. 117448, 1986. 1986; Temin, In: Gene Transfer, Kucherlapati, R ed., New York, Plenum Press, pp. 149-188, 1986). Preferred gene therapy vectors are generally viral vectors.

In using viral delivery systems, one will desire to purify the virion sufficiently to render it essentially free of undesirable contaminants, such as defective interfering viral particles or endotoxins and other pyrogens such that it will not cause any unwanted reactions in the cell, animal or individual receiving the vector construct. A preferred means of purifying the vector involves the use of buoyant density gradients, such as cesium chloride gradient centrifugation.

DNA viruses used as gene vectors include the papovaviruses (e.g., simian virus 40, bovine papilloma virus, and polyoma) (Ridgeway, pp 467492, 1988; Baichwal and Sugden, 1986) and adenoviruses (Ridgeway, 1988; Baichwal and Sugden, 1986).

One of the preferred methods for in vivo delivery involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include, but is not limited to, constructs containing adenovirus sequences sufficient to (a) support packaging of the construct and (b) to express an antisense or a sense polynucleotide that has been cloned therein.

Generation and propagation of adenovirus vectors that are replication deficient depend on a unique helper cell line, designated 293, which is transformed from human embryonic kidney cells by Ad5 DNA fragments and constitutively expresses E1 proteins (Graham et al., J. Gen. Viral., 36:59-72, 1977). Since the E3 region is dispensable from the adenovirus genome (Jones and Shenk, Cell, 13:181488, 1978), the current adenovirus vectors, with the help of 293 cells, carry foreign DNA in either the E1, the E3, or both regions (Graham and Prevec, In: Methods in Molecular Biology: Gene Transfer and Expression Protocol, E. J. Murray ed., Humana Press, Clifton, N.J., 7:109-128, 1991).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As discussed, the preferred helper cell line is 293. Racher et al., (Biotechnol. Tech, 9:169474, 1995) disclosed improved methods for culturing 293 cells and propagating adenovirus.

Adenovirus vectors have been used in eukaryotic gene expression (Levrero et al., Gene, 101:195-202, 1991; Gomez-Foix et al., J. Biol. Chem., 267:25129-25134, 1992) and vaccine development (Grunhaus and Horwitz, 1992; Graham and Prevec, 1991). Animal studies have suggested that recombinant adenovirus could be used for gene therapy (Stratford-Perricaudet and Perricaudet, In: Human Gene Transfer, O. Cohen-Haguenauer et al., eds. John Libbey Eurotext, France, pp. 51-61, 1991; Stratford-Perricaudet et al., Hum. Gene Ther. 1:241-256, 1990; Rich et al., Hum, Gene. Ther. 4:461-476, 1993). Studies in administering recombinant adenovirus to different tissues include trachea instillation (Rosenfeld et al., Science, 252: 431434, 1991; Rosenfeld et al., Cell, 68: 143-155, 1992), muscle injection (Bacot et al., Nature, 361:647-650, 1993), peripheral intravenous injections (Herz and Gerard, Proc., Natl. Acad. Sci, USA, 90:2812-2816, 1993) and stereotactic innoculation into the brain (Le Gal La Salle et al., Science, 259:988-990, 1993).

Other gene transfer vectors may be constructed from retroviruses, (Coffin, In: Virology, Fields et al., eds., Raven Press, New York, pp. 14374500, 1990.) The retroviral genome contains three genes, gag, poi, and env. that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences, and also are required for integration in the host cell genome (Coffin, 1990).

In order to construct a retroviral vector, a nucleic acid encoding protein of interest is inserted into the viral genome in the place of certain viral sequences to produce a virus that is replication-defective. In order to produce virions, a packaging cell line containing the gag, poi, and env genes, but without the LTR and packaging components, is constructed (Mann et al., Cell, 33:153459, 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin. 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., Virology, 67:242.248, 1975).

Other viral vectors may be employed as expression constructs. Vectors derived from viruses such as vaccinia virus (Ridgeway, 1988; Baichwal and Sugden, 1986; Coupar et al., Gene 68:1-10, 1988), adeno associated virus (AAV) (Ridgeway, 1988; Baichwal and Sugden, 1986; Hennonat and Muzycska, Proc. Natl. Acad. Sci. USA, 81: 6466-6470, 1984), and herpes viruses may be employed. They offer several attractive features for various mammalian cells (Friedmann, Science, 244:1275-1281., 1989; Ridgeway, 1988; Baichwal and Sugden, 1986; Coupar et al., 1988; Horwich et al., J. Virol., 64:642-650, 1990),

Several non-viral methods for the transfer of expression constructs into cultured mammalian cells also are contemplated by the present invention. These include calcium phosphate precipitation (Graham and van der Eb, Virology, 52:456467, 1973; Chen and Okayama, Mol. Cell. Biol., 7:2745-2752, 1987; Rippe et al., Mol. Cell Biol, 10: 689-695, 1990; DEAE dextran (Gopal, et al., Mol. Cell. Biol., 5:1188-1190, 1985), electroporation (TurKaspa et al., Mol, Cell Biol., 6:116-718, 1986; Potter et al., Proc. Natl. Acad. Sci., USA, 81: 7161-7165, 1984), direct microinjection, DNA-loaded liposomes and lipofectamine-DNA complexes, cell sonication, gene bombardment using high velocity microprojectiles and receptor-mediated transfection (Wu and Wu, J. Biol. Chem. 262:44294432, 1987; Wu and Wu, Biochemistry, 27:887492, 1988). Some of these techniques may be successfully adapted for in vivo or ex vivo use.

In a further embodiment, the expression construct may be entrapped in a liposome. Liposome-mediated nucleic acid delivery and expression of foreign DNA in vitro has been very successful. Wong et al., (Gene, 10:87-94, 1980) demonstrated the feasibility of liposome-mediated delivery and expression of foreign DNA in cultured chick embryo, HeLa, and hepatoma cells. Nicolau et al., (Methods Enzymol, 149:157-176, 1987) accomplished successful liposome-mediated gene transfer in rats after intravenous injection.

Where clinical applications are contemplated, it may be necessary to prepare pharmaceutical compositions—expression vectors, virus stocks, proteins, synthetic peptide conjugates, antibodies and drugs—in a form appropriate for the intended application. Generally, this will entail preparing compositions that are essentially free of impurities that could be harmful to humans or animals.

One generally will desire to employ appropriate salts and buffers in the compositions disclosed herein. Buffers also are employed when recombinant cells are introduced into a patient. Aqueous compositions of the present invention may comprise an effective amount of a protein, peptide, synthetic peptide conjugate, recombinant phage and/or expression vector, dissolved or dispersed in a pharmaceutically acceptable carrier or aqueous medium. Such compositions also are referred to as innocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the proteins or peptides of the present invention, its use in therapeutic compositions is contemplated. Supplementary active ingredients also can be incorporated into the compositions.

The active compositions of the present invention may include classic pharmaceutical preparations. Administration of these compositions according to the present invention are via any common route so long as the target tissue is available via that route. This includes oral, nasal, buccal, rectal, vaginal or topical. Alternatively, administration may be by orthotopic, intradermal, subcutaneous, intramuscular, intraperitoneal, intraarterial or intravenous injection. Such compositions normally would be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques which yield a powder of the active ingredient plus any additional desired ingredient from a previously sterile-filtered solution thereof.

In certain embodiments, therapeutic agents may be attached to a targeting peptide or synthetic peptide conjugate for selective delivery to, for example, white adipose tissue. Agents or factors suitable for use may include any chemical compound that induces apoptosis, cell death, cell stasis and/or anti-angiogenesis.

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., Bcl.sub.XL, Bcl.sub.W, Bcl.sub.S, Mcl-1, A1, Bfl4) or counteract Bcl-2 function and promote cell death (e.g., Bax Bak Bik, Bim, Bid, Bad, Harakiri).

Non-limiting examples of pro-apoptosis therapeutic peptides and/or agents contemplated within the scope of the present invention include gramicidin, magainin, mellitin, defensin, cecropin, (KLAKLAK)₂ (SEQ ID NO:32), (KLAKKLA)₂ (SEQ ID NO:33), (KAAKKAA)₂ (SEQ ID NO:34) or (KLGKKLG)₃ (SEQ ID NO:35).

In certain embodiments, administration of targeting peptides attached to anti-angiogenic agents are provided (synthetic peptide conjugates). Exemplary anti-angiogenic agents include, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazoie, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP470, endostatin, paclitaxel accutin, angiostatin, cidofovir, vincristine, bleomycin. AGM-1470, platelet factor 4 or minocycline.

Proliferation of tumors cells relies heavily on extensive tumor vascularization, which accompanies cancer progression. Thus, inhibition of new blood vessel formation with anti-angiogenic agents and targeted destruction of existing blood vessels have been introduced as an effective and relatively non-toxic approach to tumor treatment. (Arap et al., Science 279:377-380, 1998; Arap et al. Curr. Opin. Oncol. 10:560-565, 1998; Ellerby et al., Nature Med. 5:1032-1038, 1999). A variety of anti-angiogenic agents and/or blood vessel inhibitors are known, (E.g., Folkman, In: Cancer: Principles and Practice, eds, De Vita et al., pp. 3075-3085, Lippincott-Raven, New York, 1997; Eliceiri and Cheresh, Curr. Opin. Cell. Biol. 13, 563-568, 2001).

White fat represents a unique tissue that, like tumors, can quickly proliferate and expand (Wasserman, In: Handbook of Physiology, eds. Renold and Cahill, pp. 87-100, American Physiological Society, Washington, D.C., 1965; Cinti. Eat. Weight. Disord. 5:132-142, 2000). Studies of adipose tissue reveal that it is highly vascularized. Multiple capillaries make contacts with every adipocyte, suggesting the importance of the vasculature for maintenance of the fat mass (Crandall et al., Microcirculation 4:211-232, 1997). A hypothesis underlying the present application is that adipose tissue proliferation might rely on angiogenesis similarly to tumors. If so destruction of fat neovasculature could prevent the development of obesity, whereas targeting existing adipose blood vessels could potentially result in fat regression. Methods of use of adipose targeting peptides may include induction of weight loss, treatment of obesity and/or treatment of HIV related lipodystrophy.

Chemotherapeutic (cytotoxic) agents coupled with targeting peptides and/or the synthetic peptide conjugates described herein of potential use include, but are not limited to, 5-fluorouracil, bleomycin, busulfan, camptothecin carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic-agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences” 15.sup.th ed., pp 1035-1038 and 1570-1580, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

Alkylating agents are drugs that directly interact with genomic. DNA to prevent cells from proliferating. This category of chemotherapeutic drugs represents agents that affect all phases of the cell cycle, that is, they are not phase-specific. An alkylating agent, may include, but is not limited to, a nitrogen mustard, an ethylenimene, a methylmelamine, an alkyl sultanate, a nitrosourea or a triazines. They include but are not limited to: busulfan, chlorambucil, cisplatin, cyclophosphamide (cytoxan), dacarbazine, ifosfamide, mechlorethainine (mustargen), and melphalan.

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

Natural products generally refer to compounds originally isolated from a natural source, and identified as having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate dating a specific phase during the cell cycle. Mitotic inhibitors include, for example, docetaxel, etoposide (VP15), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

Certain antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of cytotoxic antibiotics include, but are not limited to, bleomycin, dactinomycin, daunorubicin, doxorubicin (Adriamycin), plicamycin (mithramycin) and idarubicin.

Miscellaneous cytotoxic agents that do not fall into the previous categories include, but are not limited to, platinum coordination complexes, anthracenediones, substituted ureas, methyl hydrazine derivatives, amsacrine, L-asparaginase and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N methylhydrazine, MIH). These examples are not limiting and it is contemplated that any known cytotoxic, cytostatic or cytocidal agent may be attached to targeting peptides and administered to a targeted organ, tissue or cell type within the scope of the invention.

The skilled artisan is directed to “Remington's Pharmaceutical Sciences” 15th Edition, chapter 33, and in particular to pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Office of Biologics standards.

### EXAMPLES

### Example 1

### ABL-1 Metabolism

While not wishing to be bound by any particular theory, metabolic breakdown of ABL-1 likely occurs by hydrolytic cleavage of the peptide via endopeptidases within the L-amino acids of the cyclic targeting portion of the compound. The targeting sequence is likely susceptible to cleavage by plasma kallikrein, an abundant peptidohydrolytic enzyme in blood which is involved in Factor XII and Plasminogen activation. (ExPASy PeptideCutter Tool, Swiss Institute of Bioinformatics). Kallikrein preferentially cleaves the Arg-Xaa peptide bond (i.e. C-terminal ‘R’ in CKGGRAKDC (SEQ ID NO:1)). Upon cleavage of an internal peptide bond, or reduction of the cysteine (C1-C2) disulfide, the peptide has an exposed N-terminus, making it susceptible to aminopeptidases, which remove the N-terminal residues, as well as endopeptidase cleavage at other residues, particularly lysines.

While the ABL-1 peptide is targeted to Prohibitin-expressing cells, metabolites that retain the _{D}[KLAKLAK]₂ (SEQ ID NO: 31) sequence could act as untargeted apoptosis-inducing peptides.

The _{D}[KLAKLAK]₂ (SEQ ID NO: 31) therapeutic peptide may have limited cytotoxicity at the desired site of activity (i.e., in situ), due to the inability of the highly positively charged peptide to disrupt eukaryotic plasma membranes. However, in the kidneys, uptake by proximal tubule cells is likely, in particular if metabolites containing N-terminal L-amino acid fragments act as substrates for receptor-mediated endocytosis and active transport. Once in the endosome, lysine-rich peptides such as the ABL-1 metabolites may be capable of endosomal escape, placing the KLAKLAK (SEQ ID NO: 36) peptide in the cytoplasm where it can then disrupt mitochondrial membranes and induce apoptosis.

### Example 2

### ABL-2a

(SEQ ID NO: 37)
_{D}C-_{D}K-G-G-_{D}R-_{D}A-_{D}K-_{D}D-_{D}C-G-G-_{D}K-_{D}L-_{D}A-_{D}K-_{D}L-_{D}A-_{D}K-_{D}K-_{D}L-_{D}A-_{D}K-_{D}L-
_{D}A-_{D}K [C1-C2 disulfide]

For ABL-2a the cyclic Prohibitin targeting sequence has been replaced with its D-amino acid analog. This is likely to have the effect of greatly increasing the plasma half-life of ABL-2a compared to ABL-1, as well as balancing the metabolic degradation rate of the two segments, thus reducing the concentration of ‘untargeted’ _{D}(ICLAKLAK)-(SEQ ID NO:38) containing metabolites in the body.

ABL-2a metabolites will likely be cleared by the body in a very different manner from the ABL-1 metabolites. If particular metabolites of ABL-1 act as substrates for active transport by renal proximal tubule cells, reabsorption of ABL-2a in the kidneys should be significantly reduced and clearance of the intact peptide promoted. ABL-2b:

(SEQ ID NO: 39)
_{D}C-_{D}D-_{D}K-_{D}A-_{D}R-G-G-_{D}K-_{D}C-G-G-_{D}K-_{D}L-_{D}A-_{D}K-_{D}L-_{D}A-_{D}K-_{D}K-_{D}L-_{D}A-_{D}K-_{D}L-_{D}A-_{D}K
[C1-C2 disulfide]

For ABL-2b the retro-inverse sequence of the Prohibitin targeting peptide KGGRAKD (SEQ ID NO: 40), namely _{D}(DKARGGK) (SEQ ID NO: 41) has been used. By inverting the stereochemistry and reversing the N-to-C arrangement of the peptide, the topology of the peptide's side-chains may be preserved while achieving increased resistance to proteolysis. This peptidometic approach has been described previously, and special consideration is necessary regarding the symmetry of the peptide side-chains when applying the strategy to cyclic peptides [P. M. Fischer, Curr. Protein Peptide Sci. (2003) 4, 339-356]. Since the N-terminal cysteine is cyclized, reversing the orientation of the terminal residue is unlikely to alter the biological activity.

### Example 3

### ABL-3

(SEQ ID NO: 42)
C-K-G-G-R-A-K-D-C-G-G-_{D}K-_{D}L-_{D}A-_{D}K-_{D}L-_{D}A-_{D}K-_{L}R-_{L}L-_{D}A-_{D}K-_{D}L-_{D}A-_{D}K
[C1-C2 disulfide]

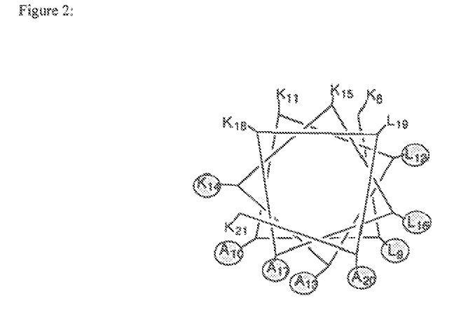

ABL-3 and ABL-4 are designed such that the breakdown of the apoptotic peptide matches that of the prohibitin targeting peptide, ABL-3 and ABL-4 are, therefore, designed result in metabolites that contain fragments of KLAKLAK (SEQ ID NO: 36) that are too short to induce apoptosis of renal tubule cells. ABL-3 and AMA incorporate L-amino acids near the center of the KLAKLAKKLAKLAK (SEQ ID NO: 29) sequence. The (KLAKLAK) (SEQ ID NO: 32) motif requires at the minimum to promote apoptosis. Thus a single cleavage site within the sequence is likely to be sufficient to eliminate the apoptotic peptide's activity. It is further envisaged to insert an endopeptidase cleavage site into the sequence without disrupting its amphipathic helical structure. Plesniak et al., Protein Science (2004), 13:1988-1996, examined the structural interaction of a the peptide, CNGRC-GG_{D}(KLAKLAK)₂, (SEQ ID NO: 43) with a model micellar membrane by NMR shown in FIG. 2.

ABL-3 introduces two L-amino acids, Arg-Leu (R-L) sites at position 15-16 in the K₈L₉A₁₀K ₁₁L₁₂AK₁₄K₁₅L₁₆A₁₇K ₁₈L₁₉A₂₀K₂₁ (SEQ ID NO: 29) peptide, ABL-3 was also designed with a single L-Arg replacing D-Lys and L-Leu replacing D-Leu. This approach is likely to disrupt the helical structure and may promote endopeptidase cleavage.

### Example 4

### ABL-4

(SEQ ID NO: 44)
C-K-G-G-R-A-K-D-C-G-G-_{D}K-_{D}L-_{D}A-_{D}K-_{D}L-_{D}A-_{D}K-G-_{L}R-_{L}A-_{L}K-_{D}K-_{D}L-_{D}A-
_{D}K-_{D}L-_{D}A-_{D}K [C1-C2 disulfide]

ABL-4 incorporates a longer stretch of L-amino acids from the peptide targeting epitope. Gly-Arg-Ala-Lys (GRAK) (SEQ ID NO: 45), inserted between the two KLAKLAK (SEQ ID NO: 36) segments. These occupy positions 15-18 in the model shown in FIG. 2. Since each helical turn is associated with about 3.5 amino acid residues, the GRAK segment should add an extra turn to the helix. While nearly preserving the original amphipathic arrangement of the adjacent KLAKLAK sequences. The Arg-Ala peptide bond is likely to be susceptible to hydrolytic cleavage by plasma kallikrein (and other endopeptidases) at about the same rate at the Arg-Ala (R-A) site in the targeting peptide. The original left-handed helical structure is not likely to be disrupted.

### Example 5

### ABL-5

(SEQ ID NO: 46)
C-K-G-G-R-A-K-D-C-G-G-_{D}K-_{D}Fx-_{D}A-_{D}K-_{L}Fx-_{D}A-_{D}K-_{D}K-_{L}Fx-_{D}A-_{D}K-_{D}Fx-_{D}A-
_{D}K [C1-C2 disulfide]

Horton et al., J. Med. Chem. 2009, 52, 3293-3299 performed a systematic in vitro optimization of the sub-cellular localization of KLAKLAK-like (SEQ ID NO: 36) peptides through sequence modification, ABL-5 is designed to have increased hydrophobicity to increase increases mitochondrial localization. A diasteriomeric peptide _{D,L}-(KExAKExAK)₂ (SEQ ID NO: 47) was prepared in which the 5^{th} and 9^{th} residues were replaced with L-analogs to disrupt alpha helicity. This compound was found to have similar cytotoxicity against HeLa cells, but greatly reduced hemolytic activity.

ABL-5 is likely to require a lower therapeutic dose and broaden the therapeutic window. Cellular uptake in the renal proximal tubules may occur by a different mechanism (endocytosis) than in the targeted adipose vascular cells (receptor-mediated membrane transport). Endosornal escape is dependent on the concentration of positively charged residues on the peptide, so reducing the overall concentration may facilitate sequestration of ABL-5 into lysosomes in the proximal tubules and subsequent elimination.

### Example 6

### ABL-6 and ABL-7

(SEQ ID NO: 48)
C-K-G-G-R-A-K-D-C-G-(PEG)ₙ-G-_{D}K-_{D}L-_{D}A-_{D}K-_{D}L-_{D}A-_{D}K-_{D}K-_{D}L-_{D}A-_{D}K-_{D}L-
_{D}A-_{D}K
(SEQ ID NO: 49)
ABL-7 (PEG)ₙ-C-K-G-G-R-A-K-D-C-G-G-_{D}K-_{D}L-_{D}A-_{D}K-_{D}L-_{D}A-_{D}K-_{D}K-_{D}L-_{D}A-
_{D}K-_{D}L-_{D}A-_{D}K

PEGylation is a widely employed strategy to enhance the in vivo activity of drugs. Hydrophilic polyethylene glycol) (PEG) polymers can be attached to various sites on peptides, proteins or small molecule drugs. PEGylation can block sensitive site on proteins from enzymatic action, or can serve to increase molecular weight of a drug molecule and thereby decrease renal elimination. Several general strategies exist for PEGylation of peptides and proteins—1) site specific modification with a single, high-molecular weight (e.g. 30 kDa) PEG polymer; 2) non-specific incorporation of low molecular weight PEG onto multiple reactive sites on a protein (e.g. at Lys residues); or 3) direct incorporation of a PEG “spacer” into a peptide backbone during synthesis.

In the case of ABL-1, the invention envisages PEGylating the arginine in the KGGRAKD (SEQ ID NO: 13) targeting peptide to block endopeptidase action. PEGylation of the KLAKLAK (SEQ ID no: 36) apoptotic sequence at the Lys side-chains or C-terminus could slow uptake by renal tubules and increase in vivo half-life, however it may also inhibit the mitochondrial membrane disrupting activity, which relies on a high density of positive amino acids.

Incorporating a PEG spacer into the center of the peptide sequence, as envisaged in ABL-6, is likely to increase the molecular weight of the compound while having the best chance of retaining the biological activity of both the targeting and the apoptotic peptide segments. Furthermore, as the cyclic targeting peptide is degraded by peptidases, the (PEG)ₙ-G_{D}(KLAKLAK)₂ (SEG ID NO: 50) should exhibit reduced uptake by renal tubule cells and overall slower renal clearance, possibly reducing toxicity. A PEG molecular weight in the range of 200 to 1000 Mw allows for balance with the targeting and apoptotic peptide segments, which are ˜1.000 and ˜1500 Mw respectively, ensuring that the active peptide components don't become buried in a globular PEG polymer. Recently, a targeted liposome using the CKGGRAKDC (SEQ ID NO:1) with a 2000 Mw PEG spacer was reported, and uptake by adipose-derived endothelial cells observed. Hossen et al., J. Controlled Release 147 (2010) 261-265.

Attachment of a higher molecular weight (2000 Da to 40,000 Da) PEG polymer to another portion of the peptide near the Arg residue, such as the N-terminus, as envisaged ABL-7, could block endopeptidase action and promote passage through the kidneys by glomeridar filtration, increasing circulating half-life.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

A10 references cited above are incorporated herein by reference to the extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A synthetic peptide conjugate capable of targeting and ablating adipose tissue vasculature comprising:
a. at least one targeting peptide;
b. at least one therapeutic peptide; and
c. a linker operatively linking the targeting and the therapeutic peptide.

2. The synthetic peptide conjugate of claim 1 wherein the peptide has 1, 2, 3, 4, 5, 6, 7, 8, 9, 10 or more of either or both of the targeting or therapeutic peptides.

3. The synthetic peptide conjugate of claim 1 selected from the group consisting of _{D}(CKGGRAKDC)-GG-_{D}(KLAKLAKKLAKLAK) (SEQ ID NO: 28); _{D}(CDKARGGKC)-GG-_{D}(KLAKLAKKLAKLAK) (SEQ ID NO: 3); CKGGRAKDC-GG-_{D}(KLAKLAK)-RL-_{D}(AKLAK) (SEQ ID NO: 4); CKGGRAKDC-GG-_{D}(KLAKLAK)-GRAK-_{D}(KLAKLAK) (SEQ ID NO: 5); CKGGRAKDC-GG-_{D,L}-(KFxAKFxAKKFxAKFxAK) (wherein Fx can be a cyclohexylalanine) (SEQ ID NO: 6); CKGGRAKDC-G-(PEG)-G-_{D}(KLAKLAKKLAKLAK) (SEQ ID NO: 7); and (PEG)-CKGGRAKDC-GG-_{D}(KLAKLAKKLAKLAK) (SEQ ID NO: 27).

4. The synthetic peptide conjugate of claim 1 wherein the targeting peptide is selected from the group consisting of TRNTGNI (SEQ ID NO:8), FDGQDRS (SEQ ID NO:9); WGPKRL: (SEQ ID NO:10); WGESRL (SEQ ID NO:11); VMGSVTG (SEQ ID NO:12), KGGRAKD (SEQILS NO:13), RGEVLWS (SEQ ID NO:14), TREVHRS (SEQ ID NO:15); HGQTVRP (SEQ ID NO:16); CKGGRAKDC (SEQ ID NO:17); or substantially similar variants thereof.

5. The synthetic peptide conjugate of claim 1 wherein the targeting peptide comprises an endopeptidase cleavage site.

6. The synthetic peptide conjugate of claim 1 wherein the targeting peptide is _{D}C-_{D}K-G-G-_{D}R-_{D}A-_{D}K-_{D}D-_{D}C (SEQ ID NO: 18).

7. The synthetic peptide conjugate of claim 1 wherein the targeting peptide is _{D}C-_{D}D-_{D}K-DA-_{D}R-G-G-_{D}K-_{D}C (SEQ ID NO: 19).

8. The synthetic peptide conjugate of claim 1 wherein the linker comprises a polymer.

9. The synthetic peptide conjugate of claim 1 wherein the linker comprises at least one amino acid.

10. The synthetic peptide conjugate of claim 1 wherein the linker comprises at least one amino acid and at least one polymer.

11. The synthetic peptide conjugate of claim 8 wherein the polymer is polyethylene glycol.

12. The synthetic peptide conjugate of claim 1 wherein the therapeutic peptide is selected from the group consisting of KLAKLAKKLAKLAK (SEQ. ID NO: 29), (KLAKKLA)₂ (SEQ ID NO: 33), (KAAKKAA)₂ (SEQ ID NO: 34) or (KLGKKLG)₃ (SEQ ID NO: 35) or a peptide substantially similar thereto.

13. The synthetic peptide conjugate of claim 1 wherein the therapeutic peptide is selected from the group consisting of _{D}K-_{D}L-_{D}A-_{D}K-_{D}L-_{D}A-_{D}K-_{D}K-_{D}L-_{D}A-_{D}K-_{D}L-_{D}A-_{D}K (SEQ ID NO: 22); _{D}K_{D}L-_{D}A-A-_{D}L-_{D}A-_{D}K-_{L}R-_{L}L-_{D}A-_{D}K-_{D}L-_{D}A-_{D}K (SEQ ID NO: 23); _{D}K-_{D}L-_{D}A-_{D}K_{D}L-_{D}A-_{D}K-G-_{L}R-_{L}A-_{D}K-_{D}K-_{D}L-_{D}A-_{D}K-_{D}L-_{D}A-_{D}K, (SEQ ID NO: 24); and _{D}K-_{D}Fx-_{D}A-_{D}K-_{L}Fx-_{D}A-_{D}K-_{D}X-_{D}Fx-_{D}A-_{D}K_{D}Fx-_{D}A-_{D}K (SEQ ID NO: 25), wherein the Fx is a modified or non-natural amino acid.

14. The synthetic peptide conjugate of claim 13 wherein Fx is cyclohexylalanine.

15. The synthetic peptide conjugate of claim 11 comprising at least one polymer selected from the group consisting of PEG 100, PEG 200, PEG 300, PEG 400, PEG 500, PEG 600, PEG 700, PEG 800, PEG 900, PEG 1000, PEG 1100, PEG 1200, PEG 1300, PEG 1400, PEG 1500 PEG 1600, PEG 1700, PEG 1800, PEG2000, PEG 3000, PEG 4000, PEG 10000, PEG 20000, PEG 40000 or mixtures thereof.

16. A method for treating or preventing obesity and/or a metabolic disorder in a patient comprising providing a patient in need thereof with a therapeutically effective amount of any of the synthetic peptide conjugates claimed in claim 1.

17. The method of claim 16 wherein the synthetic peptide conjugate is selected from the group consisting of: _{D}(CKGGRAKDC)-GG-_{D}(KLAKLAKKLAKLAK) (SEQ ID NO: 28); _{D}(CDKARGGKC)-GG-_{D}(KLAKLAKKLAKLAK) (SEQ ID NO: 3); CKGGRAKDC-GG-_{D}(KLAKLAK)-RL-_{D}(AKLAK) (SEQ ID NO: 4); CKGGRAKDC G-G-_{D}(KLAKLAK)-GRAK-_{D}(KLAKLAK) (SEQ ID NO: 5); CKGGRAKDC-GG-_{D,L}-(KFxAKFxAKKFxAKFxAK) (wherein Fx can be a cyclohexylalanine) (SEQ ID NO: 6); CKGGRAKDC-G-(PEG)₂₇-G-_{D}(KLAKLAKKIAKLAK) (SEQ ID NO: 30) and (PEG)-CKGGRAKDC-GG-D(KLAKLAKKLAKLAK) (SEQ ID NO: 27).
